(19) United States
(12) Patent Application Publication
(10) Pub. No.: US 2015/0148304 A1
(43) Pub. Date: May 28, 2015

(54) Novel Peptides and Analogs for Use in the Treatment of Macrophage Activation Syndrome

(71) Applicant: Soligenix, Inc., Princeton, NJ (US)

(72) Inventors: Oreola Donini, Coquitlam (CA); Christopher Schaber, Princeton, NJ (US); Kevin Horgan, Philadelphia, PA (US)

(21) Appl. No.: 14/523,341

(22) Filed: Oct. 24, 2014

Related U.S. Application Data
(60) Provisional application No. 61/895,351, filed on Oct. 24, 2013.

(51) Int. Cl. A61K 38/08; A61K 31/485; C07K 7/06

(52) U.S. Cl. CPC A61K 38/08; C07K 7/06; A61K 31/485
USPC 514/21.8

(57) ABSTRACT

Innate Defense Regulators (IDRs) interact with intracellular signaling events and modulate the innate defense response. Whereas much of the initial work with the IDRs focused on their role in fighting infection, recent results in animal models of chemotherapy- or radiation-induced mucositis and wound healing suggest that IDRs can be beneficial during the responses to a broader range of damage-inducing agents beyond pathogens. RIVPA (SEQ ID NO. 5), has demonstrated safety in humans and efficacy in animal models of fractionated radiation-induced and chemotherapy-induced oral mucositis, in models of chemotherapy induced damage to the gastro-intestinal tract and in models of local and systemic Gram-positive and Gram-negative infection in immunocompetent and immunocompromised hosts. Based on this information, we propose the use of RIVPA (SEQ ID NO. 5) and/or other IDRs (Table 1) as a novel treatment for Macrophage Activation Syndrome.

### RELATED APPLICATIONS

This application claims priority from U.S. Provisional Application No. 61/895,351, filed on Oct. 24, 2013, the contents of which are hereby incorporated by reference herein.

### INTRODUCTION

### Macrophage Activation Syndrome

Macrophage activation syndrome (MAS) is a serious complication of childhood systemic inflammatory disorders that is thought to be caused by excessive activation and proliferation of T lymphocytes and macrophages. MAS is a life-threatening complication of rheumatic disease that, for unknown reasons, occurs much more frequently in individuals with systemic juvenile idiopathic arthritis (SJIA) and in those with adult-onset Still disease. MAS is characterized by pancytopenia, liver insufficiency, coagulopathy, and neurologic symptoms and is thought to be caused by the activation and uncontrolled proliferation of T lymphocytes and well-differentiated macrophages, leading to widespread hemophagocytosis and cytokine overproduction.

MAS is characterized by a highly stimulated but ineffective immune response. However, its pathogenesis is poorly understood and has many similarities with that of the other forms of hemophagocytic lymphohistiocytosis (HLH). HLH is not a single disease but is a hyperinflammatory syndrome that can occur in association with various underlying genetic and acquired conditions. The best known form is familial HLH (FHLH), which is characterized by a severe impairment of lymphocyte cytotoxicity. Recent studies have shown that MUNC 13-4 polymorphisms are associated with macrophage activation syndrome in some patients with SJIA.

The cytotoxic activity of natural killer (NK) and CM⁺ T lymphocytes is mediated by the release of cytolytic granules, which contain perforin, granzymes, and other serinelike proteases, to the target cells. Several independent genetic loci related to the release of cytolytic granules have been associated with FHLH, and mutations at this level cause a severe impairment of cytotoxic function of NK cells and cytotoxic T lymphocytes (CTLs) in patients with FHLH. Through mechanisms that have not yet been well elucidated, this impairment in cytotoxic function leads to an excessive expansion and activation of cytotoxic cells, with hypersecretion of proinflammatory cytokines such as interferon (IFN)-γ, tumor necrosis factor (TNF)-α, interleukin (IL)-6, IL-10, and macrophage-colony-stimulating factor (M-CSF). These cytokines are produced by activated T cells and histiocytes that infiltrate all tissue and lead to tissue necrosis and organ failure.

Treatment of MAS is traditionally based on the parenteral administration of high doses of corticosteroids. However, some fatalities have been reported, even among patients treated with massive doses of corticosteroids (Grom et al. 1996; Prier et al. 1994; Stephen et al. 2001). The administration of high-dose intravenous immunoglobulins, cyclophosphamide, plasma exchange, and etoposide has provided conflicting results.

The use of cyclosporin A (CyA) was considered based on its proven benefit in the management of familial hemophagocytic lymphohistiocytosis (FHLH). CyA was found to be effective in severe or corticosteroid-resistant macrophage activation syndrome (Ravelli et al. 2001; Mouy et al. 1996; Ravelli et al. 1996). In some patients, this drug exerted a “switch-off” effect on the disease process, leading to quick disappearance of fever and improvement of laboratory abnormalities within 12-24 hours (Ravelli et al. 2001). Because of the distinctive efficacy of CyA, some authors have proposed using this drug as the first-line treatment for macrophage activation syndrome occurring in childhood systemic inflammatory disorders (Ravelli et al. 2001; Mouy et al. 1996).

Increased production of TNF in the acute phase of MAS has suggested the use of TNF-α inhibitors as potential therapeutic agents. However, although Prahalad et al. reported the efficacy of etanercept in a boy who developed macrophage activation syndrome, (Prahalad et al. 2001) other investigators have observed the onset of macrophage activation syndrome in patients with systemic juvenile idiopathic arthritis (SJIA) who were treated with etanercept (Prahalad et al. 2001; Ramanan et al. 2003). Similarly, Lurati et al reported the onset of macrophage activation syndrome in a patient with systemic juvenile idiopathic arthritis during treatment with the recombinant interleukin (IL)-1 receptor-antagonist anakinra (Lurati et al. 2005). Macrophage activation syndrome has also been reported in a patient with adult-onset Still disease who was receiving anakinra (Fitzgerald et al. 2005).

Although the association between macrophage activation syndrome onset and treatment with etanercept or anakinra may be coincidental and not causal, the above-mentioned observations suggest that inhibition of tumor necrosis factor (TNF) or IL-1 does not prevent macrophage activation syndrome. Moreover, although macrophage activation syndrome-like symptoms are almost completely prevented by elimination of CD8⁺ T cells or by neutralization of INF-λ in perforin-deficient mice, in the animal model of hemophagocytic lymphohistiocytosis (HLH), inhibition of IL-1 or TNF provides only mild alleviation of the symptoms.

Despite these observations, several cases of SJIA-associated macrophage activation syndrome dramatically benefiting from anakinra after inadequate response to corticosteroids and cyclosporin have now been reported (Kelly et al. 2008; Miettunen et al. 2011; Nigrovic et al. 2011; Bruck et al. 2011; Record et al. 2011). For those severely ill children, IL-1 blockade has been remarkably effective in a relatively brief time frame.

Other forms of HLH not associated with rheumatic diseases usually require more aggressive treatment: for instance, children younger than 1 year in whom FHLH is suspected and all patients with severe signs and symptoms are candidates for combination therapy with dexamethasone, cyclosporin A, and etoposide. Etoposide has been shown to improve prognosis for Epstein-Barr virus (EBV)-related HLH; its effectiveness may be explained by inhibition of synthesis of EBV nuclear antigen. Whether HLH therapeutic protocols are suitable for use in children with macrophage activation syndrome associated with rheumatic diseases is unclear.

Despite aggressive treatment, long-term disease-free survival in patients with FHLH can be reached only after stem cell transplantation.

### Innate Defenses and TLRs

The innate immune response is an evolutionarily conserved protective system associated with the barriers between tissues and the external environment, such as the skin, the orogastric mucosa and the airways. Providing rapid recognition and eradication of invading pathogens as well as a response to cellular damage, it is often associated with inflammatory responses and is a key contributor to the activation of adaptive immunity. Innate defenses are triggered by the binding of pathogen and/or damage associated molecules (PAMPs or DAMPs) to pattern-recognition receptors, including Toll-like receptors (TLRs). Pattern recognition receptors are found in and on many cell types, distributed throughout the body in both circulating and tissue resident compartments, and serve to provide early “danger” signals that lead to the release of non-specific antimicrobial molecules, cytokines, chemokines, and host defense proteins and peptides as well as the recruitment of immune cells (neutrophils, macrophages, monocytes) in a highly orchestrated fashion (Janeway 2002; Beutler 2003; Beutler 2004; Athman 2004; Tosi 2005; Doyle 2006; Foster 2007; Matzinger 2002). Moreover the innate immune system is directly involved in the generation of tolerance to commensal microbiota in the gastrointestinal tract and in gastrointestinal repair and immune defense (Santaolalla, 2011; Molloy 2012).

TLRs play a prominent role in innate immune responses (Takedo et al. 2005). TLRs recognize microbial components and initiate signal transduction pathways, further signaling gene expression. These gene products control innate immune responses and further instruct development of antigen-specific acquired immunity. Mammalian TLRs comprise a large family consisting of at least 11 members. TLR9 appears to be involved in the pathogenesis of several autoimmune diseases through recognition of the chromatin structure. Chloroquine is clinically used for treatment of rheumatoid arthritis and SLE, but its mechanism is unknown. Since chloroquine also blocks TLR9-dependent signaling through inhibition of the pH-dependent maturation of endosomes by acting as a basic substance to neutralize acidification in the vesicle (Hacker et al. 1998), it may act as an anti-inflammatory agent inhibiting TLR9-dependent immune responses. TLRs have been implicated in cytokine storm syndromes such as MAS. A study published by Behrens et al. (2011) showed that repeated stimulation of TLR9 in mice produced an HLH/MAS-like syndrome on a normal genetic background.

### IDRs and RIVPA

Innate Defense Regulators (IDRs) interact with intracellular signaling events and modulate the innate defense response. Whereas much of the initial work with the IDRs focused on their role in fighting infection, recent results in animal models of chemotherapy- or radiation-induced mucositis and wound healing suggest that IDRs can be beneficial during the responses to a broader range of damage-inducing agents beyond pathogens. IDRs treat and prevent infections by selectively modifying the body's innate defense responses when they are activated by PAMPs or DAMPs, without triggering associated inflammation responses (Matzinger 2002). The same mechanisms underlie positive effects seen in mucositis and wound healing models, where signaling downstream of the recognition of DAMPs is affected. RIVPA (SEQ ID NO. 5), has demonstrated safety in humans and efficacy in animal models of fractionated radiation-induced and chemotherapy-induced oral mucositis, in models of chemotherapy induced damage to the gastro-intestinal tract and in models of local and systemic Gram-positive and Gram-negative infection in immunocompetent and immunocompromised hosts. Based on this information, we propose the use of RIVPA (SEQ ID NO. 5) and/or other IDRs (Table 1) as a novel treatment for MAS.

### Morphinans Including Naltrexone

Naltrexone is an opioid receptor antagonist used primarily in the management of alcohol dependence and opioid dependence. United States Patent Publication No. 2011/0136845 by Trawick et al. describes how screening experiments to identify (+)-morphinans which inhibit TLR9 activation showed that (+)-Naltrexone resulted in an average of 51% inhibition of TLR9. Based on this information, we propose the use of Naltrexone as a novel component of treatment for MAS.

### RIVPA and Naltrexone

RIVPA (SEQ ID NO. 5) and Naltrexone modulate the innate immune system at two different levels. Naltrexone operates at the level of a specific receptor (TLR9) while RIVPA (SEQ ID NO. 5) operates downstream of all TLRs and other innate immune receptors. We propose that the combination of specific blockage and downstream modulation may be particularly effective at controlling the complex inflammatory disease environment encapsulated by MAS and related HLH disease. There is an urgent need for the development of MAS-like syndrome mitigators such as those capable of blocking TLR9 stimulation, for example Naltrexone. RIVPA (SEQ ID NO. 5) or other IDRs (Table 1), alone and in combination with Naltrexone, has the potential to be such a mitigator due to its ability to fight infection while suppressing inflammation downstream from TLR9 receptors.

### BRIEF DESCRIPTION OF DRAWINGS

FIG. 1. Impact of RIVPA (SEQ ID NO. 5) administration on blood counts (A, B), body weight (D) and cytokine release (C) in a model of macrophage activation syndrome.

FIG. 2. Impact of RIVPA (SEQ ID NO. 5) administration on blood counts (A, B) in a model of macrophage activation syndrome.

### DETAILED DESCRIPTION OF THE INVENTION

It is an object of the present invention to provide a method of treating macrophage activation syndrome (MAS) or HLH in a subject suffering from a cytokine storm, comprising administering to the patient an effective amount of:

a) a peptide comprising an amino acid sequence of up to 7 amino acids, said peptide comprising the amino acid sequence of X₁X₂X₃P (SEQ ID NO: 56), wherein:
X1 is R;
X2 is I or V, wherein X2 can be N-methylated;
X3 is I or V, wherein X3 can be N-methylated;
P is proline or a proline analogue;
wherein SEQ ID NO: 56 if the first four amino acids at the N-terminus of the peptide, or a pharmaceutical salt, ester or amide thereof and a pharmaceutically-acceptable carrier, diluent, or excipient; or

b) a peptide comprising the amino acid sequence of any of SEQ ID NOs: 5, 7, 10, 14, 17, 18, 22, 23, 24, 27, 28, 31, 34, 35, 63, 64, 66-69, 72, 76, 77 and 90 or a pharmaceutical salt, ester or amide thereof and a pharmaceutically-acceptable carrier, diluent or excipient.

It is another object of the present invention to provide a method of treating MAS or HLH in a subject suffering from a cytokine storm, wherein the peptide is SEQ ID NO: 5 or a pharmaceutical salt, ester, or amide thereof and a pharmaceutically-acceptable carrier, diluent, or excipient.

It is another object of the present invention to provide a method of treating MAS or HLH in a subject suffering from a cytokine storm, wherein the peptide is administered orally, parenterally, transdermally, intranasally.

It is yet another object of the present invention to provide a method of treating MAS or HLH in a subject suffering from a cytokine storm, wherein the effective amount of peptide administered to a subject is at least 6 mg/kg. In a preferred embodiment the effect amount of peptide administered to a subject is about 6 mg/kg to about 16 mg/kg.

It is yet another object of the present invention to provide a method of treating MAS or HLH in a subject suffering from a cytokine storm, wherein the peptide is administered to the subject in an effective dose for reducing and/or eliminating MAS or HLH symptoms.

It is still another object of the present invention to provide a method of treating MAS-like syndromes in a subject, wherein the peptide is administered in combination with a TLR9 antagonist. In a preferred embodiment the TLR9 antagonist is Naltrexone.

It is still another object of the present invention to provide a method mitigating the activation of innate immune cells and reducing the overstimulation of innate immunity in subjects suffering from MAS-like syndromes.

### A. RIVPA

### Structural Formula

The sequence of RIVPA (SEQ ID NO. 5) is: L-arginyl-L-isoleucyl-L-valyl-L-prolyl-L-alanine-amide. RIVPA (SEQ ID NO. 5) was previously referred to as IMX942. The USAN name for RIVPA (SEQ ID NO. 5) is susquetide.

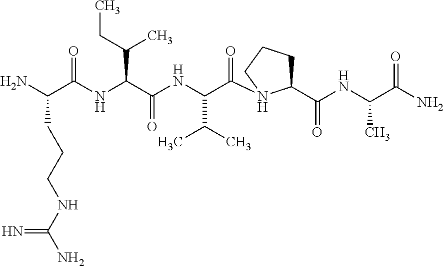 [SMILES: CCC(C)[C@H](NC(=O)[C@@H](N)CCCNC(=N)N)C(=O)N[C@H](C(=O)N1CCC[C@H]1C(=O)N[C@@H](C)C(N)=O)C(C)C]

### Formulation of the Dosage Form

The dosage form of RIVPA (SEQ ID NO. 5) is an aqueous, aseptically processed, sterile solution for injection. Each vial contains 5 mL of a 60 mg/mL solution (300 mg of RIVPA (SEQ ID NO. 5)). RIVPA (SEQ ID NO. 5) is formulated in Water for Injection and pH adjusted to a target value of 6.0. The formulation contains no excipients and has an osmolality of ˜300 mOsm/kg.

### Route of Administration

RIVPA (SEQ ID NO. 5) drug product will be diluted in sterile saline to the appropriate concentration for injection, determined on a mg/kg basis by the recipient's weight and the designated dose level. Diluted RIVPA (SEQ ID NO. 5) will be administered as an intravenous (IV) infusion in 25 ml over 4 minutes, once every second or third day.

### Pharmacology

RIVPA (SEQ ID NO. 5) binds to an intracellular adaptor protein, Sequestosome-1, also known as p62, that is involved in the efficient transmission of information during intracellular signal transduction, receptor trafficking, protein turnover (Moscat 2009) and bacterial clearance (including Salmonella [Zheng 2009], Shigella [Dupont 2009] and Listeria [Yoshikawa 2009]). p62 has recently been shown to function at a nodal position in this signaling network, interacting with MyD88 (Into 2010) and kinases and ligases downstream of TLR and Tumor Necrosis Factor (TNF) receptors (Seibenhener 2007; Moscat 2007; Kim, 2009). RIVPA (SEQ ID NO. 5) binding to p62 selectively alters its interactions with other proteins in these signaling cascades (Yu 2009). Unlike TLR-binding drugs, the binding of RIVPA (SEQ ID NO. 5) does not cause persistent activation of Nuclear Factor Kappa B (NFκB), the well-studied transcription factor associated with potentially harmful inflammatory responses. Production of pro-inflammatory cytokines such as TNFα in response to pathogen challenge is suppressed by RIVPA (SEQ ID NO. 5) treatment while the transcription factor CCAAT/enhancer binding protein β (C/EBPβ) is activated to increase expression of chemokines. In vivo studies show that RIVPA (SEQ ID NO. 5) selectively promotes monocyte and macrophage (but not neutrophil) recruitment to disease sites and speeds resolution of disease.

### Peptide Synthesis

The peptides in Table 1 were synthesized using a solid phase peptide synthesis technique.

All the required Fmoc-protected amino acids were weighed in three-fold molar excess relative to the 1 mmole of peptide desired. The amino acids were then dissolved in Dimethylformaide (DMF) (7.5 ml) to make a 3 mMol solution. The appropriate amount of Rink amide MBHA resin was weighed taking in to account the resin's substitution. The resin was then transferred into the automated synthesizer reaction vessel and was pre-soaked with Dichloromethane (DCM) for 15 minutes.

The resin was de-protected by adding 25% piperidine in DMF (30 ml) to the resin and mixing for 20 minutes. After de-protection of the resin the first coupling was made by mixing the 3 mMol amino acid solution with 4 mMol 2-(1H-benzitriazole-1-yl)-1,1,3,3-tetramethyluronium hexafluorophosphate (HBTU) and 8 mMol N,N-diisopropylethylamine (DIEPA). The solution was allowed to pre-activate for 5 minutes before being added to the resin. The amino acid was allowed to couple for 45 minutes.

After coupling the resin was thoroughly rinsed with DMF and Dimethylacetamide (DMA). The attached Fmoc protected amino acid was deprotenated in the same manner described above and the next amino acid was attached using the same coupling scheme AA:HBTU:DIEPA.

After the completion of the synthesis the peptide was cleaved from the resin with the use of a cleavage cocktail containing 97.5% Trifluoroacetic acid (TFA) and 2.5% water. The resin was allowed to swim in the cleavage cocktail for 1½ hours. The solution was then filtered by gravity using a Buchner funnel and the filtrate was collected in a 50 ml centrifugation tube. The peptide was isolated by precipitating with chilled diethyl ether. After centrifuging and decanting diethyl ether the crude peptide was washed with diethyl ether once more before being dried in a vacuum desiccator for 2 hours. The peptide was then dissolved in de-ionized water (10 ml), frozen at −80° C. and lyophilized. The dry peptide was then ready for HPLC purification.

Due to the hydrophilic nature of these peptides the diethyl ether peptide isolation did not work. Therefore a chloroform extraction was required. The TFA was evaporated and the resulting peptide residue was dissolved in 10% acetic acid (15 ml). The impurities and scavengers were removed from the acetic acid peptide solution by washing the solution twice with chloroform (30 ml). The aqueous peptide solution was then frozen at −80° C. and lyophilized resulting in a powdered peptide ready for HPLC purification.

Peptides+RIxVPA (SEQ ID NO. 33) and +RIVPAx (SEQ ID NO. 34) each contained one N-methyl amino acid. This coupling was carried out by combining the N-methyl amino acid, PyBroP and N-hydroxybenzotriazole*H2O (HOBt) and DIEPA solutions together in the RV containing the resin. After allowing to couple for 45 minutes the N-methyl amino acid was then doubled coupled to ensure complete coupling. It was observed that the coupling following the N-methyl amino acid was not fully complete. Therefore this coupling was performed using N,N,N′,N′-Tetramethyl-O-(7-azabenzotriazol-1-yl)uronium hexafluorophosphate (HATU) instead of HBTU. This still resulted in a crude peptide that typically contained two impurities totaling 30-40% of the total purity. The peptide was purified under modified HPLC conditions to isolate the pure peptide peak away from the closely eluting impurities.

R(tBg)V1KR(tBg)V2 (SEQ ID NO. 91) is an 8-residue peptide dendrimer with symmetrical branches occurring off of a fourth amino acid lysine that possesses two functional amine groups. The peptide has been synthesized with solid-phase peptide synthesis techniques, utilizing a di-Fmoc protected fourth amino acid to facilitate the coupling of the branches; followed by standard isolation and purification procedures as described above and below.

In addition, these peptides can also be synthesized with solution phase peptide synthesis techniques (Tsuda et al. 2010) and commonly known to experts in the art.

### Safety Pharmacology in Healthy Animals:

Two pilot and 2 definitive repeat-dose toxicity studies were conducted with RIVPA (SEQ ID NO. 5) in mice and cynomolgus monkeys using the intravenous (IV; slow bolus) route of administration.

Non-GLP pilot toxicology studies indicated that the maximum tolerated dose (MTD) of a single administration of RIVPA (SEQ ID NO. 5), administered as an IV injection over 30 to 60 seconds, is 88 mg/kg (actual dose) in mouse. In non-GLP pilot studies in nonhuman primates (NHP), mild clinical signs (shallow/labored respiration, decreased activity, partially closed eyes and muscle twitches) were noted in 1 or both animals after administration of 90 (1 animals), 180 (both animals) and 220 (1 animal) mg/kg RIVPA (SEQ ID NO. 5) during and shortly after dosing. These resolved within a few minutes without detectable residual effects.

The safety of multiple daily injections of RIVPA (SEQ ID NO. 5) has also been evaluated in GLP studies in mice and cynomolgus monkeys. In mouse, doses of 20, 60, or 90 mg/kg/day were given IV for 14 days. Deaths were observed at the high dose, preceded mainly by labored respiration and recumbancy. Lethality was also observed in 1 animal given 60 mg/kg but no other animals exhibited clinical signs at this dose. No test article-related mortality or clinical signs were observed at 20 mg/kg. In survivors of all groups, there was no evidence of toxicity in any organ or abnormal biochemistry or hematology. No adverse effects were observed at 20 mg/kg for 14 days.

RIVPA (SEQ ID NO. 5) at 20, 80, 160 mg/kg/day was given IV to cynomolgus monkeys for 14 days. Transient decreased activity and partially closed eyes continued to be observed during and shortly after dosing at 160 mg/kg for the first 3 days in most animals, then sporadically throughout the remaining dosing period. In all cases, these clinical signs resolved within a few minutes. No adverse effects were observed on any other measured parameter or microscopically in any tissue. The administration of RIVPA (SEQ ID NO. 5) at doses of 20 and 80 mg/kg/day did not result in any evidence of toxicity. A dose level of 80 mg/kg/day was considered to be the No-Observed-Adverse-Effect-Level (NOAEL) for this study.

No effects of RIVPA (SEQ ID NO. 5) have been observed on the central nervous system (CNS) in any study at any dose level and little or no radiolabelled RIVPA (SEQ ID NO. 5) was found in the mouse CNS at dose levels of either 20 or 90 mg/kg. No interaction was detected between RIVPA (SEQ ID NO. 5) and a battery of CNS receptors and ion channels in vitro.

A cardiovascular (CV)/pulmonary study in cynomolgus monkey using single IV doses of 20 or 80 mg/kg revealed no cardiovascular effects or changes in electrocardiogram (ECG) parameters. No respiratory effects were observed at doses of 20 or 80 mg/kg. At a dose of 80 mg/kg in this study, RIVPA (SEQ ID NO. 5) was associated with transient drooping eye lids and prostration during dosing. At 220 mg/kg, the administration of RIVPA (SEQ ID NO. 5) was associated with transient, severe clinical signs such as drooping eye lids, tremor, prostration, paleness, convulsion and collapse. In 1 animal, the high dose caused a marked reduction in respiratory rate followed by bradycardia, hypotension and death.

Overall, the NOAEL is considered to be 80 mg/kg/day for cynomolgus monkeys since transient clinical signs were limited to a single study and occurred in only 2 instances of the 98 administrations of the drug at this dose level.

No carcinogenicity, mutagenicity or reproductive toxicity studies have been conducted with RIVPA (SEQ ID NO. 5).

The effect of RIVPA (SEQ ID NO. 5) on the innate defense system is highly selective. Consistent with these findings, no changes were observed in immune-related organ weights, histopathology, hematology and clinical chemistry during mouse and NHP 14-day toxicity studies. In the latter study, no effect on T-cell, B-cell or NK-cell counts was observed after 14 days of intravenous RIVPA (SEQ ID NO. 5) dosing in the NHP. RIVPA (SEQ ID NO. 5) did not promote the proliferation of either mouse or human normal blood cells in vitro, nor of primary human leukemia cells in vitro. Collectively, there is no indication of a potential for RIVPA (SEQ ID NO. 5) to cause immunotoxicity or non-specific immune activation. No hyperactivation or suppression of adaptive immune responses, or other impact on the phenotypes of cells associated with adaptive immunity, has been detected following RIVPA (SEQ ID NO. 5) administration.

In summary, the major toxicological finding was an acute-onset respiratory depression, accompanied by labored breathing, recumbency and transient decreased activity. At its most severe, the acute toxicity resulted in death. Clinical signs were all reversible when dosing was discontinued and animals were observed to recover within minutes, with no subsequent adverse sequellae of clinical symptoms or toxicological findings. A cardiovascular/pulmonary safety pharmacology study in nonhuman primates confirmed no cardiac toxicity or QT prolongation was occurring.

The observed respiratory depression occurred at different dose levels in different species, and was not predicted by allometric scaling. In particular, the mouse appeared to be the most sensitive species with acute toxicity occurring rarely at 60 mg/kg (HED: ˜5 mg/kg) and commonly at 90 mg/kg (HED: ˜7 mg/kg). In contrast in NHP (cynomologus monkey), acute toxicity occurred occasionally at 160 mg/kg (HED: ˜50 mg/kg) and consistently at 240 mg/kg (HED: ˜78 mg/kg). Further studies with RIVPA (SEQ ID NO. 5) analogs in acute mouse toxicity studies have indicated that the toxicity is related to the charge but not the specific structure (amino acid sequence) or target protein binding status of the molecule, suggesting that the acute toxicity is due to a high instantaneous concentration of a charged molecule that scales with blood volume as opposed to allometrically. Moreover, mechanistic studies in mice have Indicated that the respiratory depression is due to altered activity of the phrenic nerve.

Toxicology and PK studies in mice with alternate routes of administration (e.g., intraperitoneal or subcutaneous) have demonstrated much higher NOAELs (i.e. >200 mg/kg)

### Clinical Experience

Clinical experience with RIVPA (SEQ ID NO. 5) was obtained in a Phase 1 Study. The primary objective of the study was to determine the maximum tolerated dose (MTD) of single and repeat ascending doses of RIVPA (SEQ ID NO. 5) injectable solution following IV administration in healthy volunteers. The secondary objectives of this study included the assessment of the dose limiting toxicity (DLT), safety, PK and pharmacodynamic (PD) profiles of RIVPA (SEQ ID NO. 5) after single and repeated ascending IV doses of RIVPA (SEQ ID NO. 5). The study was divided into 2 phases: a single-ascending dose (SAD) phase and a multiple-ascending dose (MAD) phase.

### Human Safety

Single IV doses of RIVPA (SEQ ID NO. 5) were well tolerated up to the maximum tested (8 mg/kg) and daily IV doses were well tolerated up to the maximum tested (6.5 mg/kg for 7 days). There were no dose limiting toxicities (DLTs) and the MTD was not reached in either phase of the trial. There were no deaths and no clinically significant, severe, or serious Adverse Events (AEs) reported during the study. No safety concerns or significant differences in mean values or changes from baseline were observed for vital sign measurements, clinical laboratory or electrocardiogram (ECG) results between drug-treated and placebo control subjects.

### Single Ascending Dose Phase:

The incidence of TEAEs for those subjects who received RIVPA (SEQ ID NO. 5) was not dose-related and events did not occur at a clinically significant higher rate for subjects who received RIVPA (SEQ ID NO. 5) compared to those who received placebo. The most frequently reported TEAEs (observed in more than one subject who received RIVPA (SEQ ID NO. 5) and in a higher proportion (%) than placebo subjects) were study treatment procedure-related events (General Disorders and Administration Site Conditions) such as vessel puncture site haematoma, vessel puncture site reaction and vessel puncture site pain. All vessel puncture-related events were mild and determined to be unrelated to study treatment by the QI. The second most frequently reported TEAEs were Nervous System Disorders, specifically headache and dizziness; these events were only mild to moderate. All other TEAEs were reported by only 1 subject at any given dose level (maximum of 3 dose levels). No clinically significant trends in the nature or duration of TEAEs were demonstrated for any study cohort.

### Multiple Ascending Dose Phase:

The highest incidence of TEAEs was observed at the 2 highest dose levels (4.5 and 6.5 mg/kg/day). The incidence of “possibly-related” events was also higher in the 2 highest dose levels. However, due to the small sample sizes (4 subjects received active treatment in each cohort), it was not possible to conclude whether the results definitely represented a dose-response. The majority of the TEAEs were not related to study treatment and were mild in severity with only one event reported as moderate. The most frequently reported TEAEs for subjects who received RIVPA (SEQ ID NO. 5) were General Disorders and Administration Site Conditions (i.e., procedure-related events) such as vessel puncture site haematoma, vessel puncture site reaction, and vessel puncture site pain. All vessel puncture-related events were mild and judged to be unrelated to treatment. Increased alanine aminotransferase (ALT) and back pain were reported by 3 (15.0%) subjects who received RIVPA (SEQ ID NO. 5) and these events were observed by only one (10.0%) subject who received the placebo.

### Human Pharmacokinetics

Following IV administration in human subjects and consistent with findings in animal studies, RIVPA (SEQ ID NO. 5) is cleared from the circulation within minutes. In the single-dose phase of a healthy volunteer Phase 1 trial, RIVPA (SEQ ID NO. 5) was rapidly eliminated, with plasma levels decreasing to less than 10 percent of the maximum concentration (Cmax) within 9 min after the start of the 4-minute IV infusion. Following the rapid decline, a slower elimination phase was observed. The mean time of maximum concentration (Tmax) ranged between approximately 4 min and 4.8 min after the start of infusion for the dose range of 0.15 mg/kg to 8 mg/kg. Maximum plasma concentrations and total exposure levels were dose-proportional and clearance of RIVPA (SEQ ID NO. 5) from the circulation was rapid, consistent with the mouse and NHP experience.

In light of the high clearance and short elimination half-life, accumulation following daily injection was not expected to occur. In the multiple-dose Phase 1 study, RIVPA (SEQ ID NO. 5) was administered daily for 7 days and the pre-dose concentrations measured on Days 5, 6, 7, as well as on Day 8 (24 h after the start of infusion on Day 7) were below the lower limit of quantitation (LLOQ) for all of the subjects.

### Human Pharmacodynamics

In ex vivo investigations using blood samples obtained during the Phase 1 healthy human volunteer study, a number of cytokine and chemokine analytes were quantified after 4 hours of in vitro stimulation of whole blood with LPS. The inter-individual variability in analyte levels was larger than any variation in time or response to RIVPA (SEQ ID NO. 5) or placebo administration and the data were therefore self-normalized using the individual pre-dose analyte level to standardize all responses for each individual subject (the Activity Ratio). RIVPA (SEQ ID NO. 5) effects on the analyte Activity Ratios (ARs) were neither constant throughout time, nor linearly dose responsive. Nevertheless, in the dose range 0.15-2 mg/kg, there was evidence of an increase in the “anti-inflammatory status” (i.e., higher anti-inflammatory TNF RII and IL-1ra levels coupled with lower TNFα and IL-1β levels after LIDS stimulation of blood from each individual).

### B. Naltrexone

Naltrexone has been approved by the FDA in both oral and injectable extended-release formulations. Trawick et al. teaches an appropriate concentration of morphinans for injection, determined on a mL/kg basis by recipient's weight and the designated dose level.

### Pharmacology

Naltrexone and its major active metabolite 6-β-naltrexol are competitive antagonists at μ- and κ-opiod receptors, and to a lesser extent at δ-opiod receptors (Ray et al. 2010). Naltrexone is subject to significant first pass metabolism with oral bioavailability estimates ranging from 5 to 40% while being well-absorbed orally. The activity of naltrexone is believed to be due to both parent and the 6-β-naltrexol metabolite. Both parent drug and metabolites are excreted primarily by the kidney (53% to 79% of the dose); however, urinary excretion of unchanged Naltrexone accounts for less than 2% of the elimination pathway. The plasma half-lives of Naltrexone and the 6-β-naltrexol metabolite are approximately 4 hours and 13 hours, respectively. Two other minor metabolites are 2-hydroxy-3-methoxy-6-(β)-naltrexol and 2-hydroxy-3-methyl-naltrexone. Naltrexone and its metabolites are also conjugated to form additional metabolic products. Following oral administration, naltrexone undergoes rapid and nearly complete absorption with approximately 96% of the dose absorbed from the gastrointestinal tract. Peak plasma levels of both naltrexone and 6-β-naltrexol occur within one hour of dosing. Given the known pharmacokinetics of oral naltrexone, a single daily dose of 50 mg is thought to produce plasma concentrations in the clinical range, among medication compliant patients.

### Example

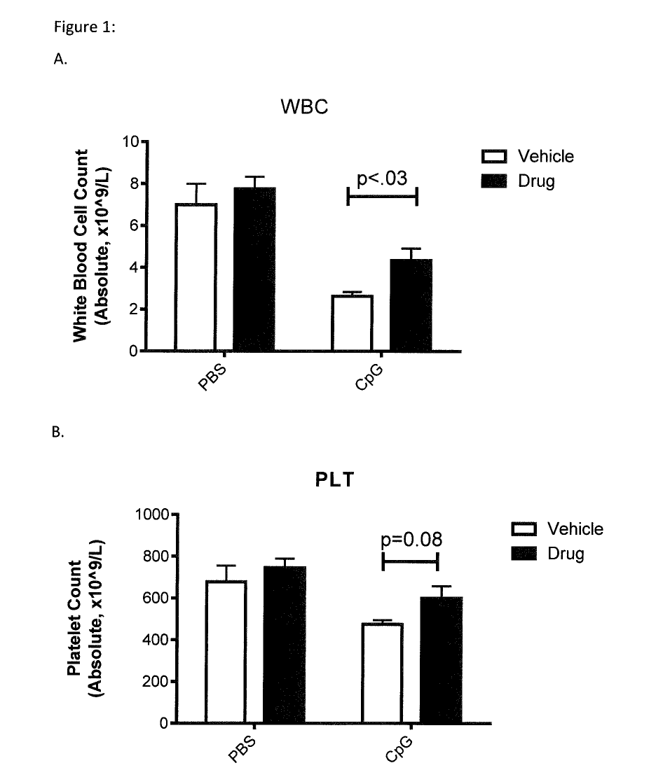

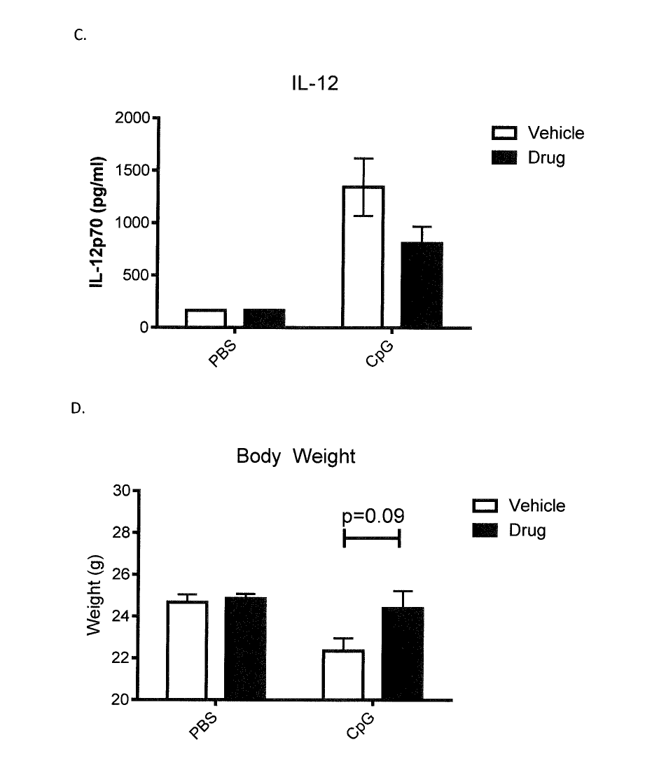

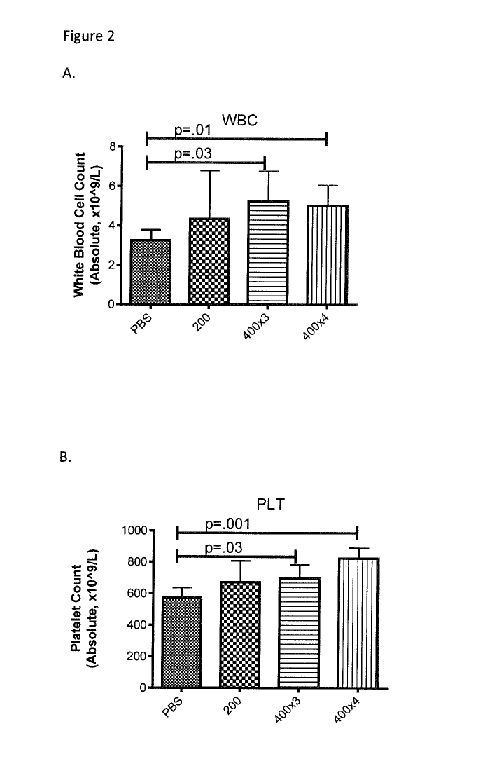

The impact of RIVPA (SEQ ID NO. 5) administration on blood counts, body weight and cytokine release was demonstrated in a model of macrophage activation syndrome (Behrens et al. 2011). Macrophage activation syndrome was simulated in 8-10 week old C57BL/6 mice by repeated administration of the TLR-9 agonist, CpG. CpG (35 μg in 200 μL) or Saline was administered intraperitoneally (IP) on days 0, 2, 4, 7 and 9. SGX94 (200 mg/kg IP) or Saline was administered on days 1, 4 and 7. Mice were observed for complete blood counts (Day 8; FIGS. 1A and B) and body weight (FIG. 1D), serum cytokines (IFNγ, IL-12 [FIG. 1C] and IL-10 on Day 10. RIVPA (SEQ ID NO. 5) significantly increased white blood cell counts and also increased platelet counts on Day 8 relative to the CpG stimulated, saline treated group. On Day 10, both decreased IL-12 levels and increased body weights was observed in the CpG stimulated and RIVPA (SEQ ID NO. 5) treated group relative to the CpG stimulated, saline-treated group. IFNγ and IL-10 levels were not significantly altered, in keeping with the general understanding of the IDR mechanism of action (Ref; Yu et al). There were no significant changes in the saline stimulated, RIVPA (SEQ ID NO. 5) treated group relative to the saline stimulated, saline treated control, as expected based on previous preclinical and clinical studies with RIVPA (SEQ ID NO. 5) and IDRs. In a repeat study, the same model was used to test administration of saline (Days 1, 4 and 7; control); RIVPA (SEQ ID NO. 5) at 200 mg/kg administered on Days 1, 4 and 7, 400 mg/kg administered on Day 1, 4 and 7 and 400 mg/kg administered on Days 1; 3, 5 and 7. In this experiment CpG (35 μg) was administered on days 0, 2, 4, 7 and 10 and no saline-stimulated controls were used. In keeping with the results from the first study a statistically significant increase in both white blood cell count and platelet count was seen with IDR treatment (FIG. 2 A and B) but no significant changes in IFNγ levels were observed.

### REFERENCES

Athman R, Philpott D. Innate immunity via Toll-like receptors and Nod proteins. Curr Opin Microbiol. 2004; 7,25-32.
Behrens E, Canna S W, Slade K, Rao S, Kreiger P A, Paessler M, Kambayashi T, Koretzky G A. Repeated TLR9 Stimulation Results in Macrophage Activation Syndrome-like Disease in Mice. J Clin. Invest. 2011; 121(6), 2264-2277.
Beutler B. Innate immunity: an overview. Mol Immunol. 2004; 40,845-59.
Beutler B, Hoebe K, Du X, Ulevitch R J. How we detect microbes and respond to them: the Toll-like receptors and their transducers. J Leukoc Biol. 2003; 74,479-85.
Bruck N, Suttorp M, Kabus M, et al. Rapid and sustained remission of systemic juvenile idiopathic arthritis-associated macrophage activation syndrome through treatment with anakinra and corticosteroids. J Clin Rheumatol. Jan. 2011; 17(1):23-7.
Doyle S L, O'Neill L A. Toll-like receptors: from the discovery of NFkappaB to new insights into transcriptional regulations in Innate immunity. Biochem Pharmacol. 2006; 72,1102-13.
Fitzgerald A A, Leclercq S A, Yan A, Homik J E, Dinarello C A. Rapid responses to anakinra in patients with refractory adult-onset Still's disease. Arthritis Rheum. June 2005; 52(6):1794-803.
Foster S L, Hargreaves D C, Medzhitov R. Gene-specific control of inflammation by TLR-induced chromatin modifications. Nature. 2007; 447,972-8.
Grom A A, Passo M. Macrophage activation syndrome in systemic juvenile rheumatoid arthritis. J Pediatr. November 1996; 129(5):630-2.
Janeway C A, Jr., Medzhitov R. Innate immune recognition. Annu Rev Immunol. 2002; 20,197-216.
Kelly A, Ramanan A V. A case of macrophage activation syndrome successfully treated with anakinra. Nat Clin Pract Rheumatol. Nov. 2008; 4(11):615-20.
Lurati A, Teruzzi B, Salmaso A, et al. Macrophagic activation syndrome (MAS) during anti-IL1 receptor therapy (anakinra) in a patient affected by systemic onset idiopathic juvenile arthritis (soJIA). Pediatr Rheumatol Online J. 2005; 3(1).
Matzinger, P. The Danger Model: A renewed sense of self. Science. 2002; 296(5566), 301-305.
Miettunen P M, Narendran A, Jayanthan A, Behrens E M, Cron R Q. Successful treatment of severe pediatric rheumatic disease-associated macrophage activation syndrome with interleukin-1 inhibition following conventional immunosuppressive therapy: case series with 12 patients. Rheumatology (Oxford). February 2011; 50(2):417-9.
Molloy M, Bouladoux N, Belkaid Y. Intestinal Microbiota: shaping local and systemic immune responses. Semin Immunol. 2012; 24(1), 58-66.
Mouy R, Stephan J L, Pillet P, Haddad E, Hubert P, Prieur A M. Efficacy of cyclosporine A in the treatment of macrophage activation syndrome in juvenile arthritis: report of five cases. J Pediatr. November 1996; 129(5):750-4.
Nigrovic P A, Mannion M, Prince F H, et al. Anakinra as first-line disease-modifying therapy in systemic juvenile idiopathic arthritis: report of forty-six patients from an international multicenter series. Arthritis Rheum. February 2011; 63(2):545-55.
Prahalad S, Bove K E, Dickens D, Lovell D J, Grom A A. Etanercept in the treatment of macrophage activation syndrome. J Rheumatol. Sep. 2001; 28(9):2120-4.
Prieur A M, Stephan J L. [Macrophage activation syndrome in rheumatic diseases in children]. Rev Rhum Ed Fr. June 1994; 61(6):447-51.
Ray L A, Chin P F, Miotto K. Naltrexone for the Treatment of Alcoholism: Clinical Findings, Mechanisms of Action, and Pharmacogenetics. CNS & Neurol. Disorders—Drug Targets 2010; 9(1):13-22.
Ramanan A V, Schneider R. Macrophage activation syndrome following initiation of etanercept in a child with systemic onset juvenile rheumatoid arthritis. J Rheumatol. February 2003; 30(2):401-3.
Ravelli A, De Benedetti F, Viola S, Martini A. Macrophage activation syndrome in systemic juvenile rheumatoid arthritis successfully treated with cyclosporine. J Pediatr. February 1996; 128(2):275-8.
Ravelli A, Viola S, De Benedetti F, Magni-Manzoni S, Tzialla C, Martini A. Dramatic efficacy of cyclosporine A in macrophage activation syndrome. Clin Exp Rheumatol. Jan.-Feb. 2001; 19(1):108.
Record J L, Beukelman T, Cron R Q. Combination therapy of abatacept and anakinra in children with refractory systemic juvenile idiopathic arthritis: a retrospective case series. J Rheumatol. January 2011; 38(1):180-1.
Santaolalla R, Fukata M, Abreu M. Innate immunity in the small intestine. Curr Opin Gastroenterol. 2011; 27(2),125-31.
Stephan J L, Kone-Paut I, Galambrun C, et al. Reactive haemophagocytic syndrome in children with inflammatory disorders. A retrospective study of 24 patients. Rheumatology (Oxford). November 2001; 40(11):1285-92.
Takedo K, Akira S. Toll-like Receptors in Innate Immunity. Internal. Imm. 2005; 17(1), 1-14.
Tosi M F. Innate immune responses to infection. J Allergy Clin Immunol. 2005; 116,241-9; quiz 50.

TABLE 1
all C-terminal amidated unless otherwise indicated****

| | | | | | | | | | | | | | | | | | | |
|---|---|---|---|---|---|---|---|---|---|---|---|---|---|---|---|---|---|---|
| SEQ ID | Notes | 1 | 2 | 3 | 4 | 5 | 6 | 7 | 8 | 9 | 10 | 11 | 12 | 13 | 14 | 15 | Length | Net charge |

| | | | | | | | | | | | | | | | | | |
|---|---|---|---|---|---|---|---|---|---|---|---|---|---|---|---|---|---|
| 1 | | + | K | S | R | I | V | P | | | | | | | | 6 | 3 |
| 2 | Ac denotes | Ac | K | S | R | I | V | P | | | | | | | | 6 | 2 |
| | acetylation | | | | | | | | | | | | | | | | |
| 3 | | | + | S | R | I | V | P | A | | | | | | | 6 | 2 |
| 4 | | | + | S | R | I | V | P | | | | | | | | 5 | 2 |
| 5 | | | | + | R | I | V | P | A | | | | | | | 5 | 2 |
| 6 | | | | + | K | I | V | P | A | | | | | | | 5 | 2 |
| 7 | *denotes | | | + | R | I | V | P | A* | | | | | | | 5 | 2 |
| | D-amino acid | | | | | | | | | | | | | | | | |
| 8 | | | | + | R | | V | P | A | | | | | | | 4 | 2 |
| 9 | | | | + | R | I | | P | A | | | | | | | 4 | 2 |
| 10 | Free acid | | | + | R | I | V | P | A | OH | | | | | | 5 | 1 |
| 11 | | | | + | R | A | V | P | A | | | | | | | 5 | 2 |
| 12 | | | + | R | R | I | V | P | A | | | | | | | 6 | 3 |
| 13 | | | | + | R | K | V | P | A | | | | | | | 5 | 3 |
| 14 | | | | + | R | I | V | P | K | | | | | | | 5 | 3 |
| 15 | | | | + | R | P | V | P | A | | | | | | | 5 | 2 |
| 16 | | | | + | R | I | P | P | A | | | | | | | 5 | 2 |
| 17 | | | | + | R | I | V | P | P | | | | | | | 5 | 2 |
| 18 | | | | + | R | I | V | P | G | G | A | | | | | 7 | 2 |
| 19 | | | + | G | G | I | V | P | A | | | | | | | 6 | 1 |
| 20 | | | | + | G | I | V | P | A | | | | | | | 5 | 1 |
| 21 | | | | + | R | G | V | P | A | | | | | | | 5 | 2 |
| 22 | | | | + | R | I | V | P | G | | | | | | | 5 | 2 |
| 23 | | | | + | R | I | V | P | S | | | | | | | 5 | 2 |
| 24 | | | | + | R | I | V | P | L | | | | | | | 5 | 2 |
| 25 | | | | + | R | H | V | P | A | | | | | | | 5 | 2? |
| 26 | | | | | + | R | I | P | V | A | | | | | | 5 | 2 |
| 27 | | | | + | R | V | I | P | A | | | | | | | 5 | 2 |
| 28 | | | | + | R | I | I | P | A | | | | | | | 5 | 2 |
| 29 | | | | | + | A | V | P | I | R | | | | | | 5 | 2 |
| 30 | | | | | | + | A | P | V | I | R | | | | | 5 | 2 |
| 31 | cyclic head- | | | | −R | I | V | P | A− | | | | | | | 5 | 1 |
| | to-tail | | | | | | | | | | | | | | | | |
| 32 | cyclic - | | | −C | R | I | V | P | A | C− | | | | | | 7 | 1 |
| | cystine link | | | | | | | | | | | | | | | | |
| 33 | x denotes | | | + | R | Ix | V | P | A | | | | | | | 5 | 2 |
| | N-methyl in | | | | | | | | | | | | | | | | |
| | backbone | | | | | | | | | | | | | | | | |
| 34 | x denotes | | | + | R | I | V | P | Ax | | | | | | | 5 | 2 |
| | N-methyl in | | | | | | | | | | | | | | | | |
| | backbone | | | | | | | | | | | | | | | | |
| 35 | | | | + | R | I | V | P | F | | | | | | | 5 | 2 |
| 36 | | | | + | Cit | I | V | P | A | | | | | | | 5 | 1 |
| 37 | | | | + | R | L | V | P | A | | | | | | | 5 | 2 |
| 38 | | | | + | H | I | V | P | A | | | | | | | 5 | 1? |
| 39 | | | + | I | R | R | V | P | A | | | | | | | 6 | 3 |
| 40 | | | | + | A | R | V | P | A | | | | | | | 5 | 2 |
| 41 | | | | + | I | R | V | P | A | | | | | | | 5 | 2 |
| 42 | | | | + | O | I | V | P | A | | | | | | | 5 | 2 |
| 43 | | | | + | S | I | V | P | A | | | | | | | 5 | 1 |
| 44 | | + | V | S | I | I | K | P | A | R | V | P | S | L | L | 13 | 3 |
| 45 | | + | K | P | A | R | V | P | S | | | | | | | 7 | 3 |
| 46 | | | + | R | | | V | P | S | L | L | | | | | 6 | 2 |
| 47 | | + | K | P | R | A | V | P | | | | | | | | 6 | 3 |
| 48 | | | + | P | A | R | V | P | | | | | | | | 5 | 2 |
| 49 | | | | + | I | R | V | P | | | | | | | | 4 | 2 |
| 50 | | | | + | R | | V | P | S | | | | | | | 8 | 2 |
| 51 | | | | + | R | | V | P | | | | | | | | 3 | 2 |
| 52 | | | | | | | + | P | S | V | P | G | S | | | 6 | 1 |
| 53 | | | + | G | L | K | H | P | S | | | | | | | 6 | 2? |
| 54 | | | | + | R | I | V | P | A | I | P | V | S | L | L | 11 | 2 |
| 55 | See Note 1 | | | | | X₁ | X₂ | P | | | | | | | | | 3 |
| 56 | See Note 2 | | | | X₁ | X₂ | X₃ | P | | | | | | | | | 4 |
| 57 | See Note 3 | | | a | X₁ | X₂ | X₃ | P | | | | | | | | | 5 |
| 58 | See Note 4 | | | | X₁ | X₂ | X₁ | P | b | | | | | | | | 5 |
| 59 | See Note 5 | | a₁ | a₂ | X₁ | X₂ | X₁ | P | | | | | | | | | 6 |
| 60 | See Note 6 | | | a | X₁ | X₂ | X₃ | P | b | | | | | | | | 6 |
| 61 | | | | + | R | I | V | P | A | C | | | | | | 6 | 2 |
| 62 | | | | + | r | r | V | P | | | | | | | | 4 | 3 |
| 63 | hydroxamic | | | + | R | I | V | P | A | HOH | | | | | | 5 | 2 |
| | acid | | | | | | | | | | | | | | | | |
| 64 | | | | + | R | I | V | P | P | A | | | | | | 6 | 2 |
| 65 | | | | + | R | I | G | P | A | | | | | | | 5 | 2 |
| 66 | | | | + | R | I | V | Pip | A | | | | | | | 5 | 2 |
| 67 | | | | + | R | I | V | Thz | A | | | | | | | 5 | 2 |
| 68 | | | | + | R | I | V | Fpro | A | | | | | | | 5 | 2 |
| 69 | | | | + | R | I | V | Dhp | A | | | | | | | 5 | 2 |
| 70 | | | | + | R | I | H | P | A | | | | | | | 5 | 2 |
| 71 | | | | + | R | I | W | P | A | | | | | | | 5 | 2 |
| 72 | | | | + | R | I | V | P | W | | | | | | | 5 | 2 |
| 73 | | | + | S | P | V | I | R | H | | | | | | | 6 | 2 |
| 74 | | | + | C | P | V | I | R | H | | | | | | | 6 | 2 |
| 75 | | | | | R | I | E | P | A | | | | | | | 5 | 1 |
| 76 | | | | + | R | I | V | P | E | | | | | | | 5 | 1 |
| 77 | | | | + | R | I | V | P | H | | | | | | | 5 | 1 |
| 78 | | | | + | R | S | V | P | A | | | | | | | 5 | 2 |
| 79 | | | + | E | R | I | V | P | A | G | | | | | | 7 | 1 |
| 80 | | | | + | K | V | I | P | S | | | | | | | 5 | 2 |
| 81 | | | | + | K | V | V | P | S | | | | | | | 5 | 2 |
| 82 | | | | | | + | K | P | R | P | | | | | | 4 | 3 |
| 83 | | | | + | R | | I | P | | | | | | | | 3 | 2 |
| 84 | | | | + | O | | V | P | | | | | | | | 3 | 2 |
| 85 | | | | + | S | | V | P | | | | | | | | 3 | 1 |
| 86 | | | | + | K | | V | P | | | | | | | | 3 | 2 |
| 87 | | | | + | R | | R | P | | | | | | | | 3 | 3 |
| 88 | | | | + | G | | V | P | | | | | | | | 3 | 1 |
| 89 | | | | | + | K | H | P | | | | | | | | 3 | 2 |
| 90 | *denotes | | | | R | I | V | P | A | Y* | | | | | | 6 | 2 |
| | D- amino acid | | | | | | | | | | | | | | | | |
| 91 | R(tBg)V is | | | | R | tBg | V | K | R | Bg | V− | | | | | 8 | |
| | linked via | | | | | | | | | | | | | | | | |
| | the side | | | | | | | | | | | | | | | | |
| | chain amino | | | | | | | | | | | | | | | | |
| | group of | | | | | | | | | | | | | | | | |
| | lysine to | | | | | | | | | | | | | | | | |
| | the valine | | | | | | | | | | | | | | | | |
| | of another | | | | | | | | | | | | | | | | |
| | R(tBg)V− | | | | | | | | | | | | | | | | |
| 92 | mp2 = | | | | R | I | V | mp2 | A | NH₂ | | | | | | 5 | |
| | 4-Amino-1- | | | | | | | | | | | | | | | | |
| | methyl-1H- | | | | | | | | | | | | | | | | |
| | pyrrole-2- | | | | | | | | | | | | | | | | |
| | carboxylic | | | | | | | | | | | | | | | | |
| | acid | | | | | | | | | | | | | | | | |

**% DPPIV Activity (Saline), where control is 100% activity (saline or vehicle alone without the peptide). About 75% or less activity relative to saline control is desirable.
****OH indicates the free acid form of the peptide. Ac indicates acetylated. O indicated Ornithine, Cit indicated Citrulline, tBG = tert-butyl glycine, mp2 = 4-Amino-1-methyl-1H-pyrrole-2-carboxylic acid
x indicates NMe backbone (versus amide backbone).
Note 1 of Table 1:
X₁ is selected from the group consisting of K, H, R, S, T, O, Cit, Hei, Dab, Dpr or glycine based compounds with basic funcational groups on the N-terminal (e.g., Nlys), hSer, Val(betaOH), X₂ is selected from the group consisting of V, I, K, P, and H including an isolated peptide of up to 10 amino acids comprising an amino acid sequence of SEQ ID NO. 55.
Note 2 of Table 1:
X₁ is selected from the group consisting of K, H, R, S, T, O, Cit, Hei, Dab, Dpr or glycine based compounds with basic funcational groups on the N-terminal (e.g., Nlys), hSer, Val(betaOH), and wherein X₂ is selected from the group consisting of A, I, L, V, K, P, G, H, R, S, O, Dab, Dpr, Cit, Hci, Abu, Hva, Nle, and wherein X₂ can be N-methylated, and wherein X₃ is selected from the group consisting of I, V, P, wherein in one embodiment X₃ is not N-methylated. In one embodiment, the isolated peptide can be an amino acid sequence of up to 10 amino acids, but is not SEQ ID NO. 2 or 17.
Note 3 of Table 1
wherein X₁, X₂, and X₃ are defined as SEQ ID NO. 56, and wherein “a” is selected from the group consisting of S, P, I, R, T, L, V, A, G, K, H, O, C, M and F or an isolated peptide up to 10 amino acids comprising said sequences.
Note 4 of Table 1:
wherein X₁X₂X₃P are as defined in SEQ ID NO. 56 and “b” is selected from the group consisting of A, A*, G, S, L, F, K, C, I, V, T, Y, R, H, O and M, but in one embodiment not P. In one embodiment, the isolated peptide is a peptide of up to 10 amino acids comprising SEQ ID NO. 58 but not SEQ ID NO. 17.
Note 5 of Table 1:
wherein X₁, X₂ and X₃ are as defined in SEQ ID NO. 56 and “a” is selected from the group consisting of K, I, R, H, O, L, V, A, and G and “a₂” is selected from the group consisting of S, P, R, T, H, K, O, L, V, A, G and I. In one embodiment, “a₁” is not acetylated, or where a₁ is K, K is not acetylated or not SEQ ID NO. 2. In one embodiment, the isolated peptide comprises up to 10 amino acids comprising SEQ ID NO. 59.
Note 6 of Table 1:
wherein X₁, X₂ and X₃ are as defined in SEQ ID NO. 56 and where “a” is selected from the group consisting of S, R, K, H, O, T, I, L, V, A and G and wherein “b” is selected from the group consisting of A, V, I, L, G, K, H, R, O, S, T and F or a peptide of up to 10 amino acids comprising SEQ ID NO. 60.

What is claimed is:

1. A method of treating macrophage activation syndrome (MAS) in a subject suffering from a cytokine storm, comprising administering to the patient an effective amount of:
a) a peptide comprising an amino acid sequence of up to 7 amino acids, said peptide comprising the amino acid sequence of X1X2X3P (SEQ ID NO: 56), wherein:
X1 is R;
X2 is I or V, wherein X2 can be N-methylated;
X3 is I or V, wherein X3 can be N-methylated;
P is proline or a proline analogue;
wherein SEQ ID NO: 56 if the first four amino acids at the N-terminus of the peptide, or a pharmaceutical salt, ester or amide thereof and a pharmaceutically-acceptable carrier, diluent, or excipient; or
b) a peptide comprising the amino acid sequence of any of SEQ ID NOs: 5, 7, 10, 14, 17, 18, 22, 23, 24, 27, 28, 31, 34, 35, 63, 64, 66-69, 72, 76, 77, 90, 91 and 92 or a pharmaceutical salt, ester or amide thereof and a pharmaceutically-acceptable carrier, diluent or excipient.

2. The method of claim 1, wherein the peptide is SEQ ID NO. 5 or a pharmaceutical salt, ester, amide thereof and a pharmaceutically-acceptable carrier, diluent or excipient.

3. The method of claim 1, wherein the peptide is administered orally, subcutaneously, intramuscularly, intravenously, transdermally, intranasally, by pulmonary administration, or by osmotic pump.

4. The method of claim 1, wherein the effect amount of peptide is at least 6 mg/kg.

5. The method of claim 1, wherein the peptide is administered in combination with a TLR9 antagonist.

6. The method of claim 5, wherein the TLR9 antagonist is naltrexone.

7. A method of treating hemophagocytic lymphohistiocytosis (HLH) in a subject suffering from a cytokine storm, comprising administering to the patient an effective amount of:
a) a peptide comprising an amino acid sequence of up to 7 amino acids, said peptide comprising the amino acid sequence of X1X2X3P (SEQ ID NO: 56), wherein:
X1 is R;
X2 is I or V, wherein X2 can be N-methylated;
X3 is I or V, wherein X3 can be N-methylated;
P is proline or a proline analogue;
wherein SEQ ID NO: 56 if the first four amino acids at the N-terminus of the peptide, or a pharmaceutical salt, ester or amide thereof and a pharmaceutically-acceptable carrier, diluent, or excipient; or
b) a peptide comprising the amino acid sequence of any of SEQ ID NOs: 5, 7, 10, 14, 17, 18, 22, 23, 24, 27, 28, 31, 34, 35, 63, 64, 66-69, 72, 76, 77, 90, 91 and 92 or a pharmaceutical salt, ester or amide thereof and a pharmaceutically-acceptable carrier, diluent or excipient.

8. The method of claim 7, wherein the peptide is SEQ ID NO. 5 or a pharmaceutical salt, ester, amide thereof and a pharmaceutically-acceptable carrier, diluent or excipient.

9. The method of claim 7, wherein the peptide is administered orally, subcutaneously, intramuscularly, intravenously, transdermally, intranasally, by pulmonary administration, or by osmotic pump.

10. The method of claim 7, wherein the effect amount of peptide is at least 6 mg/kg.

11. The method of claim 7, wherein the peptide is administered in combination with a TLR9 antagonist.

12. The method of claim 11, wherein the TLR9 antagonist is naltrexone.

13. A method of mitigating the activation of innate immune cells and reducing the overstimulation of innate immunity in subjects suffering from MAS-like syndromes, comprising administering to the patient an effective amount of:
a) a peptide comprising an amino acid sequence of up to 7 amino acids, said peptide comprising the amino acid sequence of X1X2X3P (SEQ ID NO: 56), wherein:
X1 is R;
X2 is I or V, wherein X2 can be N-methylated;
X3 is I or V, wherein X3 can be N-methylated;
P is proline or a proline analogue;
wherein SEQ ID NO: 56 if the first four amino acids at the N-terminus of the peptide, or a pharmaceutical salt, ester or amide thereof and a pharmaceutically-acceptable carrier, diluent, or excipient; or
b) a peptide comprising the amino acid sequence of any of SEQ ID NOs: 5, 7, 10, 14, 17, 18, 22, 23, 24, 27, 28, 31, 34, 35, 63, 64, 66-69, 72, 76, 77, 90, 91 and 92 or a pharmaceutical salt, ester or amide thereof and a pharmaceutically-acceptable carrier, diluent or excipient.

14. The method of claim 13, wherein the peptide is SEQ ID NO. 5 or a pharmaceutical salt, ester, amide thereof and a pharmaceutically-acceptable carrier, diluent or excipient.

15. The method of claim 13, wherein the peptide is administered orally, subcutaneously, intramuscularly, intravenously, transdermally, intranasally, by pulmonary administration, or by osmotic pump.

16. The method of claim 13, wherein the effect amount of peptide is at least 6 mg/kg.

17. The method of claim 13, wherein the peptide is administered in combination with a TLR9 antagonist.

18. The method of claim 17, wherein the TLR9 antagonist is naltrexone.


SEQUENCE LISTING (92 sequences)

SEQ ID NO: 1 - protein, 6 aa, Artificial Sequence
  Immunological Peptide
KSRIVP

SEQ ID NO: 2 - protein, 6 aa, Artificial Sequence
  IMMUNOLOGICAL PEPTIDE
XSRIVP

SEQ ID NO: 3 - protein, 6 aa, Artificial Sequence
  IMMUNOLOGICAL PEPTIDE
SRIVPA

SEQ ID NO: 4 - protein, 5 aa, Artificial Sequence
  IMMUNOLOGICAL PEPTIDE
SRIVA

SEQ ID NO: 5 - protein, 5 aa, Artificial Sequence
  IMMUNOLOGICAL PEPTIDE
RIVPA

SEQ ID NO: 6 - protein, 5 aa, Artificial Sequence
  IMMUNOLOGICAL PEPTIDE
KIVPA

SEQ ID NO: 7 - protein, 5 aa, Artificial Sequence
  IMMUNOLOGICAL PEPTIDE
RIVPX

SEQ ID NO: 8 - protein, 4 aa, Artificial Sequence
  IMMUNOLOGICAL PEPTIDE
RVPA

SEQ ID NO: 9 - protein, 4 aa, Artificial Sequence
  IMMUNOLOGICAL PEPTIDE
RIPA

SEQ ID NO: 10 - protein, 5 aa, Artificial Sequence
  IMMUNOLOGICAL PEPTIDE
RIVPX

SEQ ID NO: 11 - protein, 5 aa, Artificial Sequence
  IMMUNOLOGICAL PEPTIDE
RAVPA

SEQ ID NO: 12 - protein, 6 aa, Artificial Sequence
  IMMUNOLOGICAL PEPTIDE
RRIVPA

SEQ ID NO: 13 - protein, 5 aa, Artificial Sequence
  IMMUNOLOGICAL PEPTIDE
RKVPA

SEQ ID NO: 14 - protein, 5 aa, Artificial Sequence
  IMMUNOLOGICAL PEPTIDE
RIVPK

SEQ ID NO: 15 - protein, 5 aa, Artificial Sequence
  IMMUNOLOGICAL PEPTIDE
RPVPA

SEQ ID NO: 16 - protein, 5 aa, Artificial Sequence
  IMMUNOLOGICAL PEPTIDE
RIPPA

SEQ ID NO: 17 - protein, 5 aa, Artificial Sequence
  IMMUNOLOGICAL PEPTIDE
RIVPP

SEQ ID NO: 18 - protein, 7 aa, Artificial Sequence
  IMMUNOLOGICAL PEPTIDE
RIVPGGA

SEQ ID NO: 19 - protein, 6 aa, Artificial Sequence
  IMMUNOLOGICAL PEPTIDE
GGIVPA

SEQ ID NO: 20 - protein, 5 aa, Artificial Sequence
  IMMUNOLOGICAL PEPTIDE
GIVPA

SEQ ID NO: 21 - protein, 5 aa, Artificial Sequence
  IMMUNOLOGICAL PEPTIDE
AGVPA

SEQ ID NO: 22 - protein, 5 aa, Artificial Sequence
  IMMUNOLOGICAL PEPTIDE
RIVPG

SEQ ID NO: 23 - protein, 5 aa, Artificial Sequence
  IMMUNOLOGICAL PEPTIDE
RIVPS

SEQ ID NO: 24 - protein, 5 aa, Artificial Sequence
  IMMUNOLOGICAL PEPTIDE
RIVPL

SEQ ID NO: 25 - protein, 5 aa, Artificial Sequence
  IMMUNOLOGICAL PEPTIDE
RHVPA

SEQ ID NO: 26 - protein, 5 aa, Artificial Sequence
  IMMUNOLOGICAL PEPTIDE
RIPVA

SEQ ID NO: 27 - protein, 5 aa, Artificial Sequence
  IMMUNOLOGICAL PEPTIDE
RVIPA

SEQ ID NO: 28 - protein, 5 aa, Artificial Sequence
  IMMUNOLOGICAL PEPTIDE
RIIPA

SEQ ID NO: 29 - protein, 5 aa, Artificial Sequence
  IMMUNOLOGICAL PEPTIDE
AVPIR

SEQ ID NO: 30 - protein, 5 aa, Artificial Sequence
  IMMUNOLOGICAL PEPTIDE
APVIR

SEQ ID NO: 31 - protein, 5 aa, Artificial Sequence
  IMMUNOLOGICAL PEPTIDE
RIVPA

SEQ ID NO: 32 - protein, 7 aa, Artificial Sequence
  IMMUNOLOGICAL PEPTIDE
CRIVPAC

SEQ ID NO: 33 - protein, 5 aa, Artificial Sequence
  IMMUNOLOGICAL PEPTIDE
RXVPA

SEQ ID NO: 34 - protein, 5 aa, Artificial Sequence
  IMMUNOLOGICAL PEPTIDE
RIVPX

SEQ ID NO: 35 - protein, 5 aa, Artificial Sequence
  IMMUNOLOGICAL PEPTIDE
RIVPF

SEQ ID NO: 36 - protein, 5 aa, Artificial Sequence
  IMMUNOLOGICAL PEPTIDE
XIVPA

SEQ ID NO: 37 - protein, 5 aa, Artificial Sequence
  IMMUNOLOGICAL PEPTIDE
RLVPA

SEQ ID NO: 38 - protein, 5 aa, Artificial Sequence
  IMMUNOLOGICAL PEPTIDE
HIVPA

SEQ ID NO: 39 - protein, 6 aa, Artificial Sequence
  IMMUNOLOGICAL PEPTIDE
IRRVPA

SEQ ID NO: 40 - protein, 5 aa, Artificial Sequence
  IMMUNOLOGICAL PEPTIDE
ARVPA

SEQ ID NO: 41 - protein, 5 aa, Artificial Sequence
  IMMUNOLOGICAL PEPTIDE
IRVPA

SEQ ID NO: 42 - protein, 5 aa, Artificial Sequence
  IMMUNOLOGICAL PEPTIDE
XIVPA

SEQ ID NO: 43 - protein, 5 aa, Artificial Sequence
  IMMUNOLOGICAL PEPTIDE
SIVPA

SEQ ID NO: 44 - protein, 13 aa, Artificial Sequence
  IMMUNOLOGICAL PEPTIDE
VSIIKPARVPSLL

SEQ ID NO: 45 - protein, 7 aa, Artificial Sequence
  IMMUNOLOGICAL PEPTIDE
KPARVPS

SEQ ID NO: 46 - protein, 6 aa, Artificial Sequence
  IMMUNOLOGICAL PEPTIDE
RVPSLL

SEQ ID NO: 47 - protein, 6 aa, Artificial Sequence
  IMMUNOLOGICAL PEPTIDE
KPRAVP

SEQ ID NO: 48 - protein, 5 aa, Artificial Sequence
  IMMUNOLOGICAL PEPTIDE
PARVP

SEQ ID NO: 49 - protein, 4 aa, Artificial Sequence
  IMMUNOLOGICAL PEPTIDE
IRVP

SEQ ID NO: 50 - protein, 4 aa, Artificial Sequence
  IMMUNOLOGICAL PEPTIDE
RVPS

SEQ ID NO: 51 - protein, 3 aa, Artificial Sequence
  IMMUNOLOGICAL PEPTIDE
RVP

SEQ ID NO: 52 - protein, 6 aa, Artificial Sequence
  IMMUNOLOGICAL PEPTIDE
PSVPGS

SEQ ID NO: 53 - protein, 6 aa, Artificial Sequence
  IMMUNOLOGICAL PEPTIDE
GLKHPS

SEQ ID NO: 54 - protein, 11 aa, Artificial Sequence
  IMMUNOLOGICAL PEPTIDE
RIVPAIPVSLL

SEQ ID NO: 55 - protein, 3 aa, Artificial Sequence
  IMMUNOLOGICAL PEPTIDE
XXP

SEQ ID NO: 56 - protein, 4 aa, Artificial Sequence
  IMMUNOLOGICAL PEPTIDE
XXXP

SEQ ID NO: 57 - protein, 5 aa, Artificial Sequence
  IMMUNOLOGICAL PEPTIDE
XXXXP

SEQ ID NO: 58 - protein, 5 aa, Artificial Sequence
  IMMUNOLOGICAL PEPTIDE
XXXPX

SEQ ID NO: 59 - protein, 6 aa, Artificial Sequence
  IMMUNOLOGICAL PEPTIDE
XXXXXP

SEQ ID NO: 60 - protein, 6 aa, Artificial Sequence
  IMMUNOLOGICAL PEPTIDE
XXXXPX

SEQ ID NO: 61 - protein, 6 aa, Artificial Sequence
  IMMUNOLOGICAL PEPTIDE
RIVPAC

SEQ ID NO: 62 - protein, 4 aa, Artificial Sequence
  IMMUNOLOGICAL PEPTIDE
RRVP

SEQ ID NO: 63 - protein, 5 aa, Artificial Sequence
  IMMUNOLOGICAL PEPTIDE
RIVPX

SEQ ID NO: 64 - protein, 6 aa, Artificial Sequence
  IMMUNOLOGICAL PEPTIDE
RIVPPA

SEQ ID NO: 65 - protein, 5 aa, Artificial Sequence
  IMMUNOLOGICAL PEPTIDE
RIGPA

SEQ ID NO: 66 - protein, 5 aa, Artificial Sequence
  IMMUNOLOGICAL PEPTIDE
RIVXA

SEQ ID NO: 67 - protein, 5 aa, Artificial Sequence
  IMMUNOLOGICAL PEPTIDE
RIVXA

SEQ ID NO: 68 - protein, 5 aa, Artificial Sequence
  IMMUNOLOGICAL PEPTIDE
RIVXA

SEQ ID NO: 69 - protein, 5 aa, Artificial Sequence
  IMMUNOLOGICAL PEPTIDE
RIVXA

SEQ ID NO: 70 - protein, 5 aa, Artificial Sequence
  IMMUNOLOGICAL PEPTIDE
RIHPA

SEQ ID NO: 71 - protein, 5 aa, Artificial Sequence
  IMMUNOLOGICAL PEPTIDE
RIWPA

SEQ ID NO: 72 - protein, 5 aa, Artificial Sequence
  IMMUNOLOGICAL PEPTIDE
RIVPW

SEQ ID NO: 73 - protein, 6 aa, Artificial Sequence
  IMMUNOLOGICAL PEPTIDE
SPVIRH

SEQ ID NO: 74 - protein, 6 aa, Artificial Sequence
  IMMUNOLOGICAL PEPTIDE
CPVIRH

SEQ ID NO: 75 - protein, 5 aa, Artificial Sequence
  IMMUNOLOGICAL PEPTIDE
RIEPA

SEQ ID NO: 76 - protein, 5 aa, Artificial Sequence
  IMMUNOLOGICAL PEPTIDE
RIVPE

SEQ ID NO: 77 - protein, 5 aa, Artificial Sequence
  IMMUNOLOGICAL PEPTIDE
RIVPH

SEQ ID NO: 78 - protein, 5 aa, Artificial Sequence
  IMMUNOLOGICAL PEPTIDE
RSVPA

SEQ ID NO: 79 - protein, 7 aa, Artificial Sequence
  IMMUNOLOGICAL PEPTIDE
ERIVPAG

SEQ ID NO: 80 - protein, 5 aa, Artificial Sequence
  IMMUNOLOGICAL PEPTIDE
KVIPS

SEQ ID NO: 81 - protein, 5 aa, Artificial Sequence
  IMMUNOLOGICAL PEPTIDE
KVVPS

SEQ ID NO: 82 - protein, 4 aa, Artificial Sequence
  IMMUNOLOGICAL PEPTIDE
KPRS

SEQ ID NO: 83 - protein, 3 aa, Artificial Sequence
  IMMUNOLOGICAL PEPTIDE
RIP

SEQ ID NO: 84 - protein, 3 aa, Artificial Sequence
  IMMUNOLOGICAL PEPTIDE
XVP

SEQ ID NO: 85 - protein, 3 aa, Artificial Sequence
  IMMUNOLOGICAL PEPTIDE
SVP

SEQ ID NO: 86 - protein, 3 aa, Artificial Sequence
  IMMUNOLOGICAL PEPTIDE
KVP

SEQ ID NO: 87 - protein, 3 aa, Artificial Sequence
  IMMUNOLOGICAL PEPTIDE
RRP

SEQ ID NO: 88 - protein, 3 aa, Artificial Sequence
  IMMUNOLOGICAL PEPTIDE
GVP

SEQ ID NO: 89 - protein, 3 aa, Artificial Sequence
  IMMUNOLOGICAL PEPTIDE
KHP

SEQ ID NO: 90 - protein, 6 aa, Artificial Sequence
  IMMUNOLOGICAL PEPTIDE
RIVPAX

SEQ ID NO: 91 - protein, 7 aa, Artificial Sequence
  IMMUNOLOGICAL PEPTIDE
RXVKRXV

SEQ ID NO: 92 - protein, 6 aa, Artificial Sequence
  IMMUNOLOGICAL PEPTIDE
RIVXAX